(19) United States
(12) Patent Application Publication
(10) Pub. No.: US 2006/0134056 A1
(43) Pub. Date: Jun. 22, 2006

(54) Use of alkylguanidines as cationic emulsifiers

(75) Inventors: Hannelore Fotsch, Essen (DE); Anna Howe, Moseley, VA (US); Jurgen Meyer, Essen (DE); Matthias Pascaly, Munster (DE)

Correspondence Address: Leopold Presser, Scully,;Scott, Murphy & Presser, 400 Garden City Plaza, Garden City, NY, 11530, US

(73) Assignee: GOLDSCHMIDT AG, ESSEN (DE)

(21) Appl. No.: 11/017,589

(22) Filed: Dec. 20, 2004

(51) Int. Cl. A61K 8/43; A61K 8/49

(52) U.S. Cl. 424/70.27

(57) ABSTRACT

Emulsions comprising cationic emulsifiers and optionally coemulsifiers, optionally customary auxiliaries and additives, wherein the cationic emulsifiers used are at least one compound of the general formula (I) 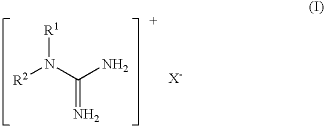 in which
R₁, R², independently of the other, is at least one radical chosen from the group H, and/or an optionally branched, optionally multiple bond-containing hydrocarbon radical, a hydroxyalkyl, alkoxy, carboxyalkyl radical, an aminoalkyl, alkylaminoalkyl, amidoalkyl, alkylamidoalkyl radical, a homo- or heterocyclic, optionally substituted aliphatic or aromatic hydrocarbon radical having 1 to 60 carbon atoms, preferably 1 to 30 carbon atoms, in particular 12 to 18 carbon atoms, with the proviso that the sum of the carbon atoms from R¹+R² is at least 12 and
X⁻ is a salt-forming anion or the hydroxyl anion are provided.

### FIELD OF THE INVENTION

The present invention relates to the use of alkylguanidines as cationic emulsifiers, and in particular for producing emulsions for cosmetic, dermatological or pharmaceutical preparations.

### BACKGROUND OF THE INVENTION

Emulsions represent an important product type in the field of cosmetic, dermatological and/or pharmaceutical preparations.

Cosmetic preparations are essentially used for skincare. Skincare in the cosmetic sense is primarily to enhance or restore the natural function of the skin as a barrier against environmental influences (e.g., dirt, chemicals, or microorganisms) and against the loss of endogenous substances (e.g., water, natural fats, or electrolytes). Impairment of this function may lead to increased absorption of toxic or allergenic substances or to attack by microorganisms, resulting in toxic or allergenic skin reactions.

A further aim of skincare is to compensate for the loss of grease and water from the skin caused by daily washing, and to achieve, or reestablish, softness and smoothness of the skin. This is particularly important when the natural regeneration ability is inadequate. Furthermore, skincare products should protect against environmental influences, in particular against sun and wind, and delay skin ageing. The sum of these skincare effects is generally summarized in cosmetics under the term “skin conditioning”.

Cosmetic preparations are also used as deodorants. Pharmaceutical topical compositions generally comprise one or more medicaments in an effective concentration. For a clear distinction between cosmetic and medical use and corresponding products, reference is made to the legal provisions of the Federal Republic of Germany (e.g., Cosmetics Directive, Foods and Drugs Act).

As a result of the continual increase in the proportion of older people in the population, for cosmetic formulations, the moisturizing effect of these preparations is increasingly coming to the fore since older people, in particular, suffer from dry skin. This moisturizing effect is achieved by cosmetically acceptable films on the skin which suppress transepidermal water loss of the skin to the atmosphere and instead increase water in the stratum corneum layer.

One way of approaching this problem using cosmetic formulations is to use care emulsions with a high oil phase content. However, when using customary nonionic or anionic emulsifiers, this may lead to such formulations having a very oily, greasy and heavy feel on the skin.

Cationic emulsions based on skin-compatible cationic emulsifiers bring about an extremely dry feel on the skin, which can be used to mask the above described effects of high lipophilic content. These facts have, in recent years, led to an increased interest in cationic emulsions in the cosmetics market being observed (see, for example, A. Paez, A. Howe, Cosmetics and Toiletries Manufacture Worldwide, May 2004, 67-71).

Combinations of an oil phase comprising, for example, one or more components, such as mineral oil, Vaseline, white oil, acetyl alcohol, or isopropyl palmitate, in combination with cationic emulsifiers, such as, for example, fatty alkyl dimethylbenzylammonium compounds, are known from the patent literature. See, for example, U.S. Pat. Nos. 3,666,690, 3,818,105 and4,137,302.

EP-B-0 058 853 discloses skincare compositions for moisturizing and conditioning the skin having improved tactile properties (feel on the skin), comprising an oil phase and quateaiy ammonium compounds of the general formula 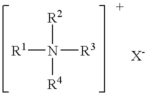
in which
R¹, R² are essentially linear alkyl chains having 16 to 22 carbon atoms,
R³, R⁴ are alkyl radicals having 1 to 3 carbon atoms, and
X⁻ is a salt-forming anion.

The preferred emulsifiers specified are quaternary ammonium compounds, such as distearyldimonium chloride.

A disadvantage of these compounds, however, is that, to achieve stable emulsions, they have to be used in relatively large amounts, typically 3 to 6% by weight, based on the formulation, of these quaternary ammonium compounds, and frequent application in these concentrations can cause allergic reactions or reactions based on hypersensitivity of the user. Further, quaternary ammonium compounds have an inadequate biodegraability by today's standards.

In addition, the dry, but in many cases, nevertheless very harsh feel on the skin, which emulsions based on these quaternary ammonium compounds have, is often a cause for complaint.

There is thus a need for emulsifiers that can produce emulsions for cosmetic, dermatological or pharmaceutical preparations which, even in considerably lower concentrations, form stable emulsions, do not cause initations on the skin and are ecologically safe and which—even when used on their own—do not cause a negatively perceived, harsh feel on the skin.

### SUMMARY OF THE INVENTION

Surprisingly, it has now been found that long-chain alkylguanidines (alkyl chain lengths of C₁₂-C₃₀) and salts thereof satisfy these requirements.

Although the use of short-chain alkylguanidines (C₁-C₁₀) in skincare products is described in the literature (see, for example, U.S. Pat. Nos. 5,723,133 and 5,939,078), only the physiological effects of these substances (as so-called active ingredients) are discussed. The use of alkylguanidines as high-performance emulsifiers is not described anywhere and could thus not have been foreseen by the person skilled in the art.

### DETAILED DESCRIPTION OF THE INVENTION

As stated above, the present invention provides long chain alkylguanidines as cationic emulsifiers for use in preparing emulsions for cosmetic, dermatological or pharmaceutical preparations.

Suitable emulsifiers according to the present invention are primarily alkylguanidines and salts thereof having a chain length from C₁₂-C₆₀, preferably C₁₂-C₃₀, of which oleyl-, stearyl-, oleyl-/stearyl-, dioleyl-, or distearylguanidinium salts in particular, are characterized by their excellent emulsifying properties in the production of cosmetic O/W emulsions.

The alkylguanidinium salts used or used according to the present invention have both good stability and also good ability to be formulated, bring about a significant effect even in low use concentrations, are nontoxic, near-natural, readily biodegradable, are very well tolerated by the skin, have high compatibility with other ingredients and can be incorporated without problems. In addition, they have a slightly antimicrobial effect.

The the long chain alkylguanidines of the present invention demonstrate their excellent emulsifier activity by virtue of the fact that, even with amounts which are considerably lower compared with the prior art, namely 0.05 to 3% by weight, preferably 0.1 to 3% by weight and in particular 0.2 to 1.5% by weight, of alkylguanidine, based on the formulation, they form stable O/W formulations, without the addition of further emulsifying substances. Higher concentrations up to 10% by weight are, however, entirely possible for specific use purposes and to additionally achieve further effects.

Furthermore, the alkylguanidines according to the present invention can also be used in virtually any desired concentration, as coemulsifiers for O/W and W/O formulations.

The feel on the skin which can achieved through the use of the alkylguanidines according to the present invention mimics that of classic cationic emulsifiers, but is in many cases less harsh and thus significantly more pleasant.

The present invention therefore provides emulsions comprising cationic emulsifiers, optionally coemulsifiers, and optionally customary auxiliaries and additives, wherein the cationic emulsifiers used are at least one compound of the general formula (I) 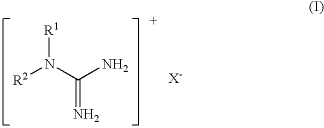
in which
R¹, R², independently of the other, is at least one radical selected from the group H, and/or an optionally branched, optionally multiple bond-containing hydrocarbon radical, a hydroxyalkyl, alkoxy, carboxyalkyl radical, an aminoalkyl, alkylaminoalkayl, amidoalkyl, alkylamidoalkyl radical, a homo- or heterocyclic, optionally substituted aliphatic or aromatic hydrocarbon radical having 1 to 60 carbon atoms, preferably 1 to 30 carbon atoms, in particular 12 to 18 carbon atoms, with the proviso that the sum of the carbon atoms from R¹+R² is at least 12, and
X⁻ is a salt-forming anion or the hydroxyl anion.

The present invention further provides the use of the emulsions for producing cosmetic, dermatological or pharmaceutical preparations.

The present invention also provides the use of the emulsions for producing cosmetic cleansing and care preparations for skin and skin appendages.

The present invention yet further provides the use of the emulsions for producing sunscreen formulations.

The present invention even further provides the use of the emulsions for producing deodorants and antiperspirants.

The present invention still further provides the use of the emulsions for producing preparations for the treatment and after-treatment of tissues based on natural or synthetic fibers, such as, in particular, for the initial finishing of textiles or, optionally in combination with quaternary compounds, as laundry softeners.

Additionally, the present invention provides the use of the emulsions for producing cleaning and care compositions for hard surfaces.

The present invention further provides the use of alkylguanidines and salts thereof in oil-in-water or in water-in-oil emulsions in a concentration of typically 0.05 to 10% by weight, preferably 0.1 to 5% by weight, in particular 0.2 to 3% by weight.

The present invention further provides the use of alkylguanidines and salts thereof as the sole emulsifiers in cosmetic and pharmaceutical oil-in-water emulsions.

The present invention further provides the use of alkylguanidines and salts thereof as coemulsifiers in cosmetic and pharmaceutical oil-in-water emulsions.

The present invention further provides the use of alkylguanidines and salts thereof as coemulsifiers in cosmetic and pharmaceutical water-in-oil emulsions.

The fatty amines used to produce the alkylguanidines used according to the invention can be prepared by known processes, e.g., by reacting fatty acids with NH₃ in the presence of catalysts to give the nitrile, and subsequent hydrogenation to give the primary amine.

Such amines are obtained from individual fatty acids or mixtures of fatty acids, such as caprylic acid, capric acid, 2-ethylhexanoic acid, lauric acid, myristic acid, palmitic acid, palmitoleic acid, isostearic acid, stearic acid, hydroxystearic acid (ricinoic acid), dihydroxysteaic acid, oleic acid, linoleic acid, petroselic acid, elaidic acid, arachic acid, behenic acid and erucic acid, gadoleic acid, and the technical-grade mixtures which form during the compressive cleavage of material fats and oils, such as oleic acid, linoleic acid, linolenic acid and, in particular, rapeseed oil fatty acid, soybean oil fatty acid, sunflower oil fatty acid, tall oil fatty acid, coconut oil. In principle, all fatty acids with a similar chain distribution are suitable. The content of unsaturated fractions in these fatty acids or fatty acid esters is, if necessary, adjusted by means of the known catalytic hydrogenation processes to the desired iodine number, or achieved by mixing completely hydrogenated fatty components with unhydrogenated fatty components.

Preference is given to using partially hydrogenated C₈₋₁₈-coconut or palm fatty acids, rapeseed oil fatty acids, sunflower oil fatty acids, soybean oil fatty acids and tall oil fatty acids, with iodine numbers in the range from about 80 to 150 and, in particular, technical-grade C₈₋₁₈-coconut fatty acids, where in some cases a choice of cis/tans isomers, such as elaidic acid-rich C_{16/18}-fatty acid cuts may be advantageous. The preferred fatty acids are commercially available products and are supplied by various companies under their respective trade names.

The alkylguanidium salts used according to the present invention accordingly have an average alkyl chain length of C₁₂-C₆₀, preferably C₁₂-C₃₀, in particular C₁₂-C₁₈.

Alkylguanidinium salts of the formula I are in equilibrium with the unprotonated alkylguanidines, the position of the equilibrium naturally being determined by the pH of the preparation.

Of suitability for the salt formation are, in principle, all cosmetically acceptable inorganic or organic mono- or polybasic acids, such as, for example, formic acid, propionic acid, heptanoic acid, caprylic acid, nonanoic acid, capric acid, undecanoic acid, lauric acid, myristic acid, palmitic acid, stearic acid, arachic acid, behenic acid, cyclopentanecarboxylic acid, cyclohexanecarboxylic acid, acrylic acid, methacrylic acid, vinylacetic acid, crotonic acid, 2-/3-/4-pentenoic acid, 2-/3-/4-/5-hexanoic acid, lauroleic acid, myristoleic acid, palmitoleic acid, oleic acid, gadoleic acid, sorbic acid, linoleic acid, linolenic acid, pivalinic acid, ethoxyacetic acid, phenylacetic acid, 2-ethylhexanoic acid, oxalic acid, glycolic acid, malonic acid, succinic acid, glutaric acid, adipic acid, pimelic acid, suberic acid, azelaic acid, sebacic acid, benzoic acid, o-/m-/p-toluic acid, salicylic acid, 3-/4-hydroxybenzoic acid, phthalic acids, or their partially or completely hydrogenated derivatives, such as hexahydro- or tetrahydrophthalic acid and mixtures thereof, in particular carbonic acid, phosphoric acid, silicic acid, acetic acid, lactic acid, tartaric acid, malic acid, citric acid. In this connection, and for the purposes of the present invention, it is also possible to use either suitable guanidine derivatives in mixtures with one another, or else mixed salts.

One way of preparing the alkylguanidines used according to the present invention is described, for example, in DE-506 282. In the process, alkylanines are guanidylated in an alcoholic solution with cyanamide in the presence of a protic acid. This reaction gives the products in the form of crystalline salts.

A further constituent of the present invention is the use of alkylguanidines and salts thereof together with one or more other emulsifiers. This use of the alkylguanidines, as coemulsifiers, is in principle possible both in oil-in-water (O/W) and also in water-in-oil (W/O) emulsions.

In particular, this use as coemulsifier relates to cosmetic skincare products in which, by virtue of the use of the alkylguanidines according to the present invention or salts thereof, firstly the emulsifier activity is enhanced and, secondly, a special feel on the skin is imparted which is particular to this class of substance. This feel on the skin can be described as very dry, but, in contrast to “classic” cationic emulsifiers is characterized in that it has a significantly less harsh effect.

If further emulsifiers are used, in addition to alkylguanidines and salts thereof suitable examples include nonionogenic surfactants from at least one of the following groups:
addition products of from 2 to 100 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group
C_{12/18}-fatty acid mono- and diesters of addition products of from 1 to 100 mol of ethylene oxide onto glycerol
glycerol mono- and diesters and sorbitan mono- and diesters of saturated and unsaturated fatty acids having 6 to 22 carbon atoms and ethylene oxide addition products thereof
alkyl mono- and oligoglycosides having 8 to 22 carbon atoms in the alkyl radical and ethoxylated analogs thereof
addition products of from 2 to 100 mol of ethylene oxide onto castor oil and/or hydrogenated castor oil
polyol and, in particular, polyglycerol esters such as, for example, polyglycerol polyricinoleate, polyglycerol-12 hydroxystearate or polyglycerol dimerate. Likewise suitable are mixtures of compounds of two or more of these classes of substances
partial esters based on linear, branched, unsaturated or saturated C_{6/22}-fatty acids, ricinoleic acid, and 12-hydroxystearic acid and glycerol, polyglycerol, pentataerytol, dipentaerythritol, sugar alcohols (e.g., sorbitol), alkyl glucosides (e.g., methyl glycoside, butyl glycoside, lauryl glucoside), and polyglucosides (e.g., cellulose)
mono-, di- and trialkyl phosphates, and mono-, di- and/or tri-PEG-alkyl phosphates and salts thereof
polysiloxane-polyether copolymers (dimethicone copolyols), such as, for example, PEG/PPG-20/6 dimethicone, PEG/PPG-20/20 dimethicone, bis-PEG/PPG-20/20 dimethicone, PEG-12 or PEG-14 dimethicone, PEG/PPG-14/4 or 14/12 or 20/20 or 18/18 or 17/18 or 15/15. Of particular suitability here are products such as bis-PEG/PPG-14/14 dimethicone (with cyclopentasiloxane:ABIL® EM 97) or in particular PEG/PPG-16/16 dimethicone (with caprylic/capric triglyceride:ABIL® Care 85)
polysiloxane-polyalkyl-polyether copolymers or corresponding derivatives, such as, for example, lauryl or acetyl dimethicone copolyols, in particular cetyl PEG/PPG-10/1 dimethicone (ABIL® EM 90)
mixed esters of pentaerythritol, fatty acids, citric acid and fatty alcohol as in DE-1 165 574 and/or mixed esters of fatty acids having 6 to 22 carbon atoms, methyl glucose and polyols, preferably glycerol or polyglycerol
polyalkylene glycol
amphoteric and zwitterionic surfactants, such as betaines
other cationic emulsifiers, such as, for example, alkyl quats, ester quats, silicone qats, and ethoxylated variants thereof.

The addition products of ethylene oxide and/or of propylene oxide onto fatty alcohols, fatty acids, alkylphenyls, glycerol mono- and diesters, and sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out.

Further emulsifiers which may be used are zwitterionic surfactants. Zwitteronic surfactants refers to those surface-active compounds which carry at least one quaternary ammonium group and at least one carboxylate and one sulfonate group in the molecule. Particularly suitable zwitterionic surfactants are the so-called betaines, such as the N-akyl-N,N-dimethylammonium glycinates, for example cocoalkyldimethylammonium glycinare, N-acylaminopropyl-N,N-dimethylammonium glycinates, for example cocoacylaminopropyldimethylammonium glycinate and 2-alkyl-3-carboxymethyl-3-hydroxyethylimidazolines having in each case 8 to 18 carbon atoms in the alkyl or acyl group, and cocoacylaminoethyl hydroxyethylcarboxymethyl glyconate. Particular preference is given to the fatty acid amide derivative known under the CTFA name Cocamidopropyl Betaine.

Likewise suitable emulsifiers are amphoteric surfactants. Amphoteric surfactants are understood as meaning those active compounds which, apart from a C_{8/18}-alkyl or -acyl group in the molecule, contain at least one flee amino group and at least one —COOH— or —SO₃H group and are capable of forming internal salts. Examples of suitable amphoteric surfactants are N-alkylglycines, N-alkylpropionic acids, N-alkylaminobutyric acids, N-alkylininodipropionic acids, N-hydroxyethyl-N-alkylamidopropylglycines, N-alkyltaurines, N-alkylsarcosines, 2-alkylaninopropionic acids and alkylaminoacetic acids having in each case about 8 to 18 carbon atoms in the alkyl group. Particularly preferred amphoteric surfactants are N-cocoalkylaminopropionate, cocoacylaminoethylaminopropionate and C_{12/18}-acylsarcosine. Besides the amphoteric emulsifiers, quaternary emulsifiers are also suitable, those of the ester quat type being particularly preferred, preferably methyl-quaternized difatty acid triethanolamine ester salts.

In addition, the inventive cationic emulsions based on alkylguanidines and salts thereof can contain customary auxiliaries and additives, such as bodying agents, thickeners, oils, waxes, UV light protection filters, antioxidants, hydrotropes, deodorant and antiperspirant active ingredients, preservatives, insect repellants, self-tanning agents, perfume oils, dyes and biogenic active ingredients.

Suitable bodying agents are primarily fatty alcohols or hydroxy fatty alcohols having 12 to 22 and preferably 16 to 18 carbon atoms, and also partial glycerides, fatty acids or hydroxy fatty acids.

Suitable thickeners are, for example, polysaccharides, in particular xanthan gum, guar, agar agar, alginates and Tyloses, carboxymethylcellulose and hydroxyethylcellulose, alkyl-modified sugar derivatives, such as, for example, cetylhydroxyethylcellulose, and also higher molecular weight polyethylene glycol monoesters and diesters of fatty acids, polyacrylamides, polyvinyl alcohol and polyvinylpyrrolidone, surfactants such as, for example, ethoxylated fatty acid glycerides, esters of fatty acids with polyols, such as, for example, pentaerythritol or trimethylolpropane, fatty alcohol ethoxylates with a narrowed homolog distbution or alkyl oligoglycosides, and electrolytes such as sodium chloride and ammonium chloride.

Suitable as the oil phase are, for example, those oil components which are known as cosmetic and pharmaceutical oil components and as components of lubricants. These include, in particular, monoesters or diesters of linear and/or branched mono- and/or dicarboxylic acids having 2 to 44 carbon atoms with linear and/or branched sated or unsaturated alcohols having 1 to 22 carbon atoms. Likewise suitable are the esterification products of aliphatic, difunctional alcohols having 2 to 36 carbon atoms with monofunctional aliphatic carboxylic acids having 1 to 22 carbon atoms. Monoesters suitable as oil components are, for example, the methyl esters and isopropyl esters of fatty acids having 12 to 22 carbon atoms, such as, for example, methyl lanate, methyl stearate, methyl oleate, methyl erucate, isopropyl palmitate, isopropyl myristate, isopropyl stearate, isopropyl oleate. Other suitable monoesters are, for example, n-butyl stearate, n-hexyl laurate, n-decyl oleate, isooctyl stearate, isononyl palmitate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, and esters which are obtainable from technical-grade aliphatic alcohol cuts and technical-grade, aliphatic carboxylic acid mixtures, e.g., esters of unsaturated fatty alcohols having 12 to 22 carbon atoms and saturated and unsaturated fatty acids having 12 to 22 carbon atoms, as are accessible from animal and vegetable fats. Also suitable, however, are naturally occurring monoester or wax ester mixtures, as are present, for example, in jojoba oil or in sperm oil.

Suitable dicarboxylic esters are, for example, di-n-butyl adipate, di-n-butyl sebacate, di(2-ethylhexyl) adipate, di(2-hexyldecyl) succinate, diisotridecyl acetate. Suitable diol esters are, for example, ethylene glycol dioleate, ethylene glycol diisotridecanoate, propylene glycol di(2-thyl hexanoate), butane diol diisostearate and neopentyl glycol dicaprylate.

Further fatty acid esters which can be used are, for example, C₁₂₋₁₅-alkyl benzoates, dicaprylyl carbonates, diethylhexyl carbonates.

Likewise suitable, as the oil component, are the fatty acid triglycerides, where, amongst these, the naturally occurring oils and fats are preferred. Suitable oil components are, for example, natural, vegetable oils, e.g. olive oil, sunflower oil, soybean oil, groundnut oil, rapeseed oil, almond oil, palm oil or else the liquid fractions of coconut oil or of palm kernel oil, and animal oils, such as, for example, neat's-foot oil, the liqud fractions of beef tallow and also synthetic triglycerides of caprylic/capric acid mixtures, triglycerides of technical-grade oleic acid or of palmitic acid/oleic acid mixtures.

In addition, hydrocarbons, in particular also liquid paraffins and isoparaffins, can be used. In addition, it is also possible to use fatty alcohols, such as oleyl alcohol or octyldodecanol, and fatty alcohol ethers, such as dicapryl ether.

Suitable silicone oils and silicone waxes are, for example, polydimethyliloxanes, cyclomethylsiloxanes, and aryl- or alyl- or alkoxy-substiued polymethylsiloxanes or cyclomethylsiloxanes.

In total, the formulations according to the invention can comprise 1 to 50% of oil phase.

UV light protection filters are understood as meaning organic substances which are able to absorb ultraviolet rays and re-emit the absorbed energy in the form of long-wave radiation, e.g., heat UVB filters may be oil-soluble or water-soluble. Examples of oil-soluble substances are:
3-benzylidenecamphor and derivatives thereof, e.g., 3-(4-methylbenzylidene)camphor
4-aminobenzoic acid derivatives, preferably 2-ethylhexyl 4-(dimethylamino)benzoate, 2-ethylhexyl 4-(dimethylamino)benzoate and amyl 4-(dimethylamino)benzoate
esters of cinnanic acid, preferably 2-ethylhexyl 4-methoxycinnamate, isopentyl 4-methoxycinnamate, 2-ethylhexyl 2-cyano-3-phenylcinnamate (octocrylene)
esters of salicylic acid, preferably 2-ethylhexyl salicylate, 4-isopropylbenzyl salicylate, homomenthyl salicylate
derivatives of benzophenone, preferably 2-hydroxy-4-methoxybenzophenone, 2-hydroxy-4-methoxy4′-methylbenzophenone, 2,2′-dihydroxy-4-methoxybenzophenone
esters of benzamalonic acid, preferably di-2-ethylhexyl 4-methoxybenzalmolonate
triazine derivatives, such as, for example, 2,4,6-trianilino(p-carbo-2′-ethyl-1′-hexyloxy)-1,3,5-triazine and octyltriazone
propane-1,3-diones, such as, for example, 1-(4-tert-butylphenyl)-3-(4′methoxyphenyl)propane-1,3-dion.

Suitable water-soluble substances are:
2-phenylbenzimidazole-5-sulfonic acid and the alkali metal, alkaline earth metal, ammonium, alkylammonium, alkanolammonium and glucammonium salts thereof
sulfonic acid derivatives of benzophenone, preferably 2-hydroxy-4-methoxybenzophenone-5-sulfonic acid and its salts
sulfonic acid derivatives of 3-benzylidenecamphor, such as, for example 4-(2-oxo-3-bornylidene-methyl)benzenesulfonic acid and 2-methyl-5-(2-oxo-3-bornylidene)sulfonic acid and salts thereof.

Suitable typical UV-A filters are, in particular, derivatives of benzoylmethane, such as, for example, 1-(4′-tertbutylphenyl)-3-(4′-methoxyphenyl)propane-1,3-dione or 1-phenyl-3-(4′-isopropylphenyl)propane-1,3 dione. The UV-A and UV-B filters can of course also be used in mixtures. Besides soluble substances, insoluble pigments, namely finely dispersed metal oxides or salts, are also suitable for this purpose, such as, for example, titanium dioxide, zinc oxide, iron oxide, aluminum oxide, cerium oxide, zirconium oxide, silicate (talc), barium sulfate and zinc stearate. Here, the particles should have an average diameter of less than 100 nm, preferably between 5 and 50 nm and in particular between 15 and 30 nm. The particles may have a spherical shape, although it is also possible to use particles which have an ellipsoidal shape or a shape which deviates in another way from the spherical form. A relatively new class of light protection filters are micronized organic pigments, such as, for example, 2,2′-methylenebis{6-(2H-benzotriazol-2-yl)-4-(1,1,3,3-tetramethylbutyl)phenol} with a particle size of less than 200 nm, which is available, for example, as a 50% strength aqueous dispersion.

Besides the two abovementioned groups of primary light protection substances, it is also possible to use secondary light protection agents of the antioxidant type, which interrupt the photochemical reaction chain which is triggered when UV radiation penetrates into the skin. Typical examples thereof are super oxide dismutase, tocopherols (vitamin A) and ascorbic acid (vitamin C). Further suitable UV light protection filters are given in the review by P. Finkel in SÖFW-Journal 122, 543 (1996).

To improve the flow behavior it is also possible to use hydrotropes, such as, for example, ethanol, isopropyl alcohol, or polyols. Polyols which are suitable here preferably have 2 to 15 carbon atoms and at least two hydroxyl groups. Typical examples are:
glycerol
alkylene glycols, such as, for example, ethylene glycol, diethylene glycol, propylene glycol, butylene glycol, hexylene glycol, and polyethylene glycols with an average molecular weight of from 100 to 1000 daltons
technical-grade oligoglycerol mixtures with a degree of self-condensation of from 1.5 to 10, such as, for example, technical-grade diglycerol mixtures with a diglycerol content of from 40 to 50% by weight
methylol compounds, such as, in particular, trimethylolethane, trimethylolpropane, trimethylolbutane, pentaerythritol and dipentaerythritol
lower alkyl glucosides, in particular those with 1 to 8 carbon atoms in the alkyl radical, such as, for example, methyl and butyl glycoside
sugar alcohols having 5 to 12 carbon atoms, such as, for example, sorbitol or mannitol
sugars having 5 to 12 carbon atoms, such as, for example, glucose or sucrose
amino sugars, such as, for example, glucamine.

Suitable deodorant active ingredients are, for example, odor concealers such as the customary perfume constituents, odor absorbers, for example the sheet silicates described in the patent laid-open specification DE-A-40 09 347, of these in particular montmorillonite, kaolinite, ilite, beidelite, nontronite, saponite, ilectorite, bentonite, smectite, and also, for example, zinc salts of ricinoleic acid. Antibacterial agents are likewise suitable to be incorporated into the oil-in-water emulsions according to the invention. Advantageous substances are, for example, 2,4,4′-trichloro-2′-hydroxydiphenyl ether (irgasan), 1,6-di(4-chlorophenylbiguanido)hexane (chlorhexidine), 3,4,4′-trichlorocarbonilide, quartanary ammonium compounds, oil of cloves, mint oil, thyme oil, triethyl citrate, farnesole (3,7,11-trimethyl-2,6,10-dodecatriene-1-ol), and the active agents described in the patent laid-open specifications DE-A-198 55 934, DE-A-37 40 186, DE-A-39 38 140, DE-A42 04 321, DE-A42 29 707, DE-A-42 29 737, DE-A42 38 081, DE-A43 09 372, DE-A43 24 219. Further customary antiperspirant active ingredients can likewise be used advantageously in the preparations according to the present invention, in particular astringents, for example basic aluminum chlorides, such as aluminum chlorohydrate (“ACH”) and aluminum-zirconium-glycine salts (“ZAG”).

Suitable preservatives are, for example, phenoxyethanol, formaldehyde solution, parabenes, pentanediol or sorbic acid.

Suitable insect repellants are N,N-diethyl-m-toluamide, 1,2-pentanediol or Insect Repellent 3535, and suitable self-tanning agents are, for example, dihydroxyacetone and erythrulose, and perfume oils which may be mentioned are mixtures of natural and synthetic fragrances. Natural fragrances are extracts from flowers (lily, lavender, rose, jasmine, neroli, ylang ylang), stems and leaves (geranium, patchouli, petit grain), frits (aniseed, coriander, caraway, juniper), fruit peels (bergamot, lemons, oranges), roots (mace, angelica, celery, cardamom, costus, iris, thyme), needles and branches (spruce, fir, pine, dwarf-pine), resins and balsams (galbanum, elemi, benzoe, myrrh, olibanum, opoponax). Also suitable are animal raw materials such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, α-isomethylionone and methyl cedryl ketone, the alcohols include anethol, citronellol, eugenol, isoeugenol, geraniol, linalool, phenylethyl alcohol and terpineol, and the hydrocarbons include predominantly the tepenes and balsams. However, preference is given to using mixtures of different fragrances which together produce a pleasing scent note. Essential oils of relatively low volatility, which are mostly used as aroma components, are also suitable as perfume oils, e.g. sage oil, camomile oil, oil of cloves, balm oil, mint oil, cinnamon leaf oil, linden flower oil, juniperberry oil, vetiver oil, olibanum oil, galbanum oil, labdanum oil and lavandine oil. Preferece is given to using bergamot oil, dihydromyecenol, lilial, lyral, citronellol, phenylethyl alcohol, α-hexylcinnamaldehyde, geraniol, benzylacetone, cyclamenaldehyde, linalool, boisambrene forte, ambroxane, indole, hedione, sandelice, lemon oil, mandarin oil, orange oil, allyl amyl glycolate, cyclovertal, lavandine oil, clary sage oil, β-damascone, geranium oil bourbon, cyclohexyl salicylate, Vertofix Coeur, ISO-Super, Fixolide NP, Evernyl, iraldein gamma, phenylacetic acid, geranyl acetate, benzyl acetate, rose oxide, romillat, irotyl and flornmat alone or in mixtures.

Dyes which can be used are the subsaces approved and suitable for cosmetic purposes, as are swlrnmae, for example, in the publication “Kosmetische Färbemitter” [Cosmetic Colorants] by the Farbstoffkommission der Deutschen Forschungsgemeinschaft, [Dyes Commission of the German Research Society], Verlag Chemie, Weinheim, 1984, pp. 81-106. These dyes are usually used in concentrations of from 0.001 to 0.1% by weight, based on the total mixture.

Biogenic active ingredients are undet as meaning, for example, tocopherol tocopherol acetate, tocopherol palmitate, ascorbic acid, deoxyribonucleic acid, retinol, bisabolol, allantoin, phytantriol, panthenol, AHA acids, amino acids, ceramides, phytosphingosine (and phytosphingosine derivatives), pseudoceramides, essential oils, plant extracts and vitamin complexes.

The following examples are provided to illustrate the present invention and to demonstrate the use of long chain alkylguanidines as a cationic emulsifier.

### PREPARATION EXAMPLES

Synthesis of laurylguanidinium acetate:

44.1 g of laurylamine (0.238 mol) and 150 ml of n-butanol were initially introduced and dissolved at 60° C. At the elevated temperature, 14.2 g of acetic acid (99.8%, 0.237 mol) were then added. A temperature of 90° C. was then established and, over the course of 3 hours, the cyanamide (11.0 g; 0.262 mol) dissolved in 50 ml of n-butanol was added dropwise. When the addition was complete, the mixture was left to after-react for 3 hours at 90° C. The solvent was then stripped off on a rotary evaporator. For purification, the crude prdduct was slurried in acetone, filtered, and the residue was washed with diethyl ether. Residual amounts of diethyl ether were removed on the rotary evaporator at reduced pressure. This gave a colorless, crystalline powder.

Lawylguanidinium acetate: ¹³C-NMR, 100 MHz, MeOD, 25° C.: δ=179.1 (1C, COOH_{AcOH}), 157.7 (1C, C_{guanidinium gr.}), 41.2 (1C, CH₂), 31.6 (1C, CH₂), 29.3 (4C, CH₂), 28.9 (2C, CH₂), 28.6 (1C, CH₂), 26.4 (1C, CH₂), 23.2 (1C, CH₂),22.3 (1C, CH_{3AcOH}), 13.1 (1C, CH₃); MADI-TOF[m/z]: 228 (M+H⁺)

Synthesis of stearylguanidinium acetate and stearylguanidinium lactate:

76.35 g of stearylamine (0.269 mol) and 150 ml of n-butanol were initially introduced and dissolved at 60° C. At the elevated temperature, 16.19 g of acetic acid (0.269 mol) or 26.95 g of lactic acid (0.269 mol) were then added. A temperature of 90° C. was then established and, over the course of 3 hours, the cyanamide (11.31 g) dissolved in 50 ml of n-butanol was added dropwise. At the end of the addition, the mixture was left to after-react for 3 hours at 90° C. The solvent was then stripped off on a rotary evaporator. For purification, the crude product was slurried in acetone, filtered and the residue was washed with diethyl ether. Residual amounts of diethyl ether were removed on the rotary evaporator at reduced pressure. This gave a white, crystalline powder.

Stearylguanidiam acetate: ¹³C-NMR, 100 MHz, MeOD, 25° C.: δ=180.1 (1C, COOH_{AcOH}), 158.8 (1C, C_{guanidinium gr.}), 42.2 (1C, CH₂), 32.8 (1C, CH₂), 30.6 (12C, CH₂), 29.7 (1C, CH₂), 27.6 (1C, CH₂), 24.6 (1C, CH₂), 23.5 (1C, CH_{3 AcOH}), 14.5 (1C, CH₃); MALDI-TOF[m/z]: 312 (M+H⁺)

Stearylguanidinium lactate: ¹³C-NMR, 100 MHz, MeOD, Toluol-d 50° C.: δ=181.7 (1C, COOH_{LacOH}), 157.9 (1C, C_{guanidinium gr.}), 68.7 (1C, CH_{LacOH}), 41.6 (1C, CH₂), 32.1 (1C, CH2), 29.5 (12C, CH₂), 29.1 (1C, CH₂), 26.9 (1C, CH₂), 22.8 (1C, CH₂), 20.9 (1C, CH_{3LacOH}), 13.8 (1C, CH₃); MALDI-TOF[m/z]:

Synthesis of distearylguanidinium lactate:

143.28 g of distearylamine (0.269 mol) and 150 ml of n-butanol were initially intoduced and dissolved at 60° C. At the elevated tempeature, 26.95 g of lactic acid (0.269 mol) were then added. A temperature of 90° C. was then established and, over the course of 3 hours, the cyanide (11.31 g) dissolved in 50 ml of n-butanol was added dropwise. At the end of the addition, the mixture was left to after-react for 3 hours at 90° C. The solvent was then stripped off on the rotary evaporator. For purification, the crude product was slurried in acetone, filtered and the residue was washed with diethyl ether. Residual amounts of diethyl ether were removed on the rotary evaporator at reducedpressu. This gave a white, crystalline powder.

Distearylguanidinium lactate: ¹³C-NMR, 100 MHz, MeOD, 50° C.: δ=182.2 (1C, COOH_{LacOH}), 157.5 (1C, C_{guanidinium gr.}), 69.2 (1C, CH_{LacOH}), 32.7 (2C, CH₂), 30.5 (28C, CH2),28.2 (2C, CH₂), 23.4 (2C, CH₂), 21.7 (1C, CH_{3,LacOH}), 14.5 (2C, CH₃); MALDI-TOF[m/z]:

### Application Examples

The examples below are intended to illustrate the subject-matter of the present invention in more detail without limiting it thereto. Examples 1 to 8 show the use of alkylguanidines as the sole emulsiers, whereas examples 9 to 18 illustrate the use of alkylguanidines as coemulsifiers. The concentrations given in all ofthe examples are % by weight.

Emulsions 1 to 4:

Emulsions 1 to 4 aim to show that typical cationic O/W creams were obtainable even with very low emulsifier concentrations of from 0.5 to 1.5% of stearylguanidine or distearylguanidine were used. Here, it was paaticulaily surprising that stable emulsions were obtainale even at emulsifier concentrations of 0.5% stearylguanidinium acetate (6 months stability at room temperature, 45° C. and freeze-thaw cycles (3×−15° C.)).

| | | Emulsion | | | |
|---|---|---|---|---|---|
| | | 1 | 2 | 3 | 4 |
| | | % | % | % | % |
| A | Glycerol stearate | 4.0 | 4.0 | 4.0 | 4.0 |
| | Stearyl alcohol | 2.0 | 2.0 | 2.0 | 2.0 |
| | Caprylic/capric triglyceride | 9.0 | 9.0 | 9.0 | 9.0 |
| | Mineral oil (30 mPas) | 8.0 | 8.0 | 8.0 | 8.0 |
| B | Glycerol | 3.0 | 3.0 | 3.0 | 3.0 |
| | Stearylguanidinium acetate | 0.5 | 1.0 | 1.5 | |
| | Distearylguanidinium acetate | | | | 1.5 |
| | Water | ad 100 | ad 100 | ad 100 | ad 100 |
| | Preservative, perfume | q.s. | q.s. | q.s. | q.s. |

Emulsions 5 to 8:

Emulsions 5 to 8 show that stable emulsions were also possible with typical other emollient combinations. In addition, emulsions 7 to 8 also illustrate the excellent emulsifying properties of other alkylguanidinium salts.

| | | Emulsion | | | |
|---|---|---|---|---|---|
| | | 5 | 6 | 7 | 8 |
| | | % | % | % | % |
| A | Glycerol stearate | 4.0 | 4.0 | 4.0 | 4.0 |
| | Stearyl alcohol | 2.0 | 2.0 | 2.0 | 2.0 |
| | Caprylic/Capric triglyceride | 8.0 | | 8.0 | |
| | C₁₂₋₁₅ alkyl benzoate | 9.0 | | 9.0 | |
| | Diethylhexyl carbonate | | 9.0 | | 9.0 |
| | Cyclopentasiloxane | | 8.0 | | 8.0 |
| B | Glycerol | 3.0 | 3.0 | 3.0 | 3.0 |
| | Stearylguanidinium acetate | 1.0 | 1.0 | | |
| | Stearylguanidinium lactate | | | 1.0 | 1.0 |
| | Water | ad 100 | ad 100 | ad 100 | ad 100 |
| | Preservative, perfume | q.s. | q.s. | q.s. | q.s. |

Emulsions 9 to 12:

Emulsions 9 to 12 show the use of alkylguamudinium salts according to the present invention as coemulsifiers in combination with typical nonionic emulsifiers for establishing a feel on the skin which was very dry, but not harsh.

| | | Emulsion | | | |
|---|---|---|---|---|---|
| | | 9 | 10 | 11 | 12 |
| | | % | % | % | % |
| A | Ceteareth-25 | 1.5 | 1.5 | 1.5 | 1.5 |
| | Stearyl alcohol | 2.0 | 2.0 | 2.0 | 2.0 |
| | Glycerol stearate | 4.0 | 4.0 | 4.0 | 4.0 |
| | Caprylic/capric triglyceride | 8.0 | 8.0 | 8.0 | 8.0 |
| | Cetearyl ethylhexanoate | 8.5 | 8.5 | 8.5 | 8.5 |
| B | Glycerol | 3.0 | 3.0 | 3.0 | 3.0 |
| | Stearylguanidinium acetate | 1.0 | | | |
| | Stearylguanidinium lactate | | 1.0 | | |
| | Laurylguanidinium acetate | | | 1.0 | |
| | Laurylguanidinium-lactate | | | | 1.0 |
| | Water | ad 100 | ad 100 | ad 100 | ad 100 |
| | Preservative, perfume | q.s. | q.s. | q.s. | q.s. |

Emulsions 13 to 15:

Emulsions 13 to 15 serve in particular to illustate the combination of alkylguanidines with silicone O/W emulsifiers. This combination was chamcteized by an excellent dry and silky feel on the skin.

| | | Emulsion | | |
|---|---|---|---|---|
| | | 13 | 14 | 15 |
| | | % | % | % |
| A | Bis-PEG/PPG-16/16 PEG/PPG 16/16 | 1.5 | 1.5 | 1.5 |
| | dimethicone; Caprylic/capric | | | |
| | triglyceride (ABIL ® Care 85) | | | |
| | Stearyl alcohol | 2.0 | 2.0 | 2.0 |
| | Glycerol stearate | 4.0 | 4.0 | 4.0 |
| | Caprylic/capric triglyceride | 8.0 | 8.0 | 8.0 |
| | Cetearyl ethylhexanoate | 8.5 | 8.5 | 8.5 |
| B | Glycerol | 3.0 | 3.0 | 3.0 |
| | Stearylguanidinium acetate | 1.0 | | |
| | Laurylguanidinium acetate | | 1.0 | |
| | Distearylguanidinium acetate | | | 1.0 |
| | Water | ad 100 | ad 100 | ad 100 |
| | Preservative, perfume | q.s. | q.s. | q.s. |

Emulsions 16 to 18:

Emulsions 16 to 18 aim to show in particular that the use of alkylguanidines as coemulsifiers was also possible in W/O emulsions.

| | | Emulsion | | |
|---|---|---|---|---|
| | | 16 | 17 | 18 |
| | | % | % | % |
| A | Cetyl PEG/PPG-10/1 dimethicone | 2.0 | 2.0 | 2.0 |
| | (ABIL ® EM 90) | | | |
| | Microcrystalline wax | 1.2 | 1.2 | 1.2 |
| | Hydrogenated castor oil | 0.8 | 0.8 | 0.8 |
| | Caprylic/capric triglyceride | 5.0 | 5.0 | 5.0 |
| | Diethylhexyl carbonate | 7.0 | 7.0 | 7.0 |
| | Cetearyl ethylhexanoate | 7.0 | 7.0 | 7.0 |
| B | NaCl | 0.5 | 0.5 | 0.5 |
| | Stearylguanidinium acetate | 0.3 | | |
| | Stearylguanidinium lactate | | 0.3 | |
| | Laurylguanidinium acetate | | | 0.3 |
| | Water | ad 100 | ad 100 | ad 100 |
| | Preservative, perfume | q.s. | q.s. | q.s. |

While the present invention has been particularly shown and described with resect to preferred embodiments thereof, it will be understood by those skilled in the art that the foregoing and othe changes in forms and details may be made without departing fiom the spirit and scope of the present invention. It is therefore intended that the present invention not be inmited to the exact forms and details described and illustrated, but fall within the spirit and scope of the appended claims.

1. A cationic skin care emulsion comprising at least one cationic emulsifier of the general formula (I) 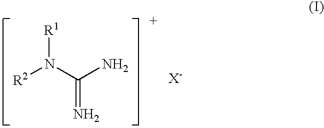
in which
R¹, R², independently of the other, is at least one radical comprising H, an optionally branched, optionally multiple bond-containing hydrocarbon radical, a hydroxyalkyl, alkoxy, carboxyalkyl radical, an aminoalkyl, alkylaminoalkyl, amidoalkyl, alkylamidoalkyl radical, a homo- or heterocyclic, optionally substituted aliphatic or aromatic hydrocarbon radical having 1 to 60 carbon atoms or mixture thereof, with the proviso that the sum of the carbon atoms from R¹+R² is at least 12, and
X⁻ is a salt-forming anion or a hydroxyl anion.

2. The cationic skin care emulsion as claimed in claim 1, wherein R¹ and R², independently of one another, are H or hydrocarbon radical having 8 to 30 carbon atoms.

3. The cationic skin care emulsion as claimed in claim 2, wherein the hydrocarbon radical has 12 to 18 carbon atoms.

4. The cationic skin care emulsion as claimed in claim 2, further comprising a non-protonated alkylguanidine.

5. The cationic skin care emulsion as claimed in claim 1, wherein R¹ and R² are hydrocarbon radicals having 12 to 18 carbon atoms.

6. The cationic skin care emulsion as claimed in claim 1, wherein R¹═H and R² is a hydrocarbon radical having 12 to 18 carbon atoms.

7. The cationic skin care emulsion as claimed in claim 1, wherein X⁻ is at least one anion of organic or inorganic mono- or polybasic acids.

8. The cationic skin care emulsion as claimed in claim 7, wherein X⁻ is one of carbonic acid, phosphoric acid, sulfonic acid, acetic acid, lactic acid, tartaric acid, malic acid or citric acid.

9. A preparation comprising at least the cationic skin care emulsion of claim 1.

10. The preparation as claimed in claim 9, further comprising a cosmetic formulation, a dermatological formulation or a pharamaceutical formulation.

11. The preparation as claimed in claim 9, further comprising a cleansing or care formulation for skin and skin appendages, a sunscreen formulation, a deodorant formulation or an antiperspirant formulation.

12. The preparation as claimed in claim 9, further comprising a tissue product based on natural or synthetic fibers.

13. The preparation as claimed in claim 9, further comprising a hard surface cleaning or care composition.

14. An oil-in-water or in water-in-oil emulsion comprising in a concentration from 0.05 to 10% by weight as a sole emulsifier or a co-emulsifiers the emulsion of claim 1.

15. A process for the preparation of an emulsion comprising mixing, in any order, a water phase with an oil phase, wherein at least one of said phases includes an emulsifying agent having the general formula (I) 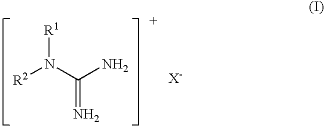
in which
R¹, R², independently of the other, is at least one radical comprising H, an optionally branched, optionally multiple bond-containing hydrocarbon radical, a hydroxyalkyl, alkoxy, carboxyalkyl radical, an aminoalkyl, alkylaminoalkyl, amidoalkyl, alkylamidoalkyl radical, a homo- or heterocyclic, optionally substituted aliphatic or aromatic hydrocarbon radical having 1 to 60 carbon atoms or mixture thereof, with the proviso that the sum of the carbon atoms fron R¹+R² is at least 12, and
X⁻ is a salt-forming anion or a hydroxyl anion.
